(19) United States
(12) Patent Application Publication
(10) Pub. No.: US 2023/0226278 A1
(43) Pub. Date: Jul. 20, 2023

(54) REUSABLE AUTO-INJECTOR DEVICE WITH REFILLABLE MEDICATION AND REPLACEABLE NEEDLE

(71) Applicant: PGMD Enterprises, LLC, Plainview, NY (US)

(72) Inventors: Gregory Puglisi, Dix Hills, NY (US); Samuel Puglisi, Dix Hills, NY (US); Edlyn Terrazas Zambrano, Phoenix, AZ (US); Katiya Beshara, Gansevoort, NY (US); Tamara Puskarevic, Lexington, MA (US); Christian Chandler Natal, Bronx, NY (US); Joshua David Bernardo, Hopkinton, MA (US)

(21) Appl. No.: 18/098,513

(22) Filed: Jan. 18, 2023

Related U.S. Application Data
(60) Provisional application No. 63/361,729, filed on Jan. 18, 2022.

(51) Int. Cl. A61M 5/24; A61M 5/315; A61M 5/32

(52) U.S. Cl. CPC A61M 5/2422; A61M 5/31515; A61M 5/3286; A61M 5/3204; A61M 5/3257

(57) ABSTRACT

An injection system and apparatus comprises a housing with a loading tray configured to accept a medication cartridge, a needle configured to transport medication from the medication cartridge to an injection site, a retractable sheath formed on an injection end of the housing, and a telescoping plunger comprising at least two telescoping sections, wherein the plunger is configured to impart downward force on the medication cartridge in order to release medication in the medication cartridge through the needle to an injection site.

### CROSS REFERENCE TO RELATED APPLICATIONS

This patent application claims the priority and benefit under 35 U.S.C. § 119(e) of U.S. Provisional Patent Application Ser. No. 63/361,729 filed Jan. 18, 2022, entitled “REUSABLE AUTO-INJECTOR DEVICE WITH REFILLABLE MEDICATION AND REPLACEABLE NEEDLE.” U.S. Provisional Patent Application Ser. No. 63/361,729 is herein incorporated by reference in its entirety.

### TECHNICAL FIELD

Embodiments are generally related to the field of medicine. Embodiments are further related to the field of injections. Embodiments are also related to injection devices. Embodiments are further related to auto-injection devices. Embodiments are also related to vaccinations, inoculations, and injectable therapeutics. Embodiments are further related to devices, systems, and methods for injectable medications that can be administered via an auto-injector device.

### BACKGROUND

There are numerous medications that are administered via injection in subcutaneous or intramuscular tissue. These medications may be administered by healthcare providers, emergency response teams, or may be self-administered. Some of the medications administered in this fashion include, but are not limited to, those for chronic conditions that are administered at regular intervals, such as biologic therapies, insulin, or human growth hormone; those designed for prophylactic use such as vaccines; and those that are rarely used but are required immediately in emergency situations, such as Epinephrine, Narcan, or Glucagon.

Administration for many of these indications is typically via an injector that houses the medication and quickly delivers the medicament into the subcutaneous or intramuscular space automatically. Many injector devices, particularly those used in the emergency setting, are designed for one-time use, containing one dose of medicament enclosed in the device. A common example is Epinephrine, which is used in the event of anaphylaxis; a life-threatening allergic emergency that can be triggered by a variety of substances including foods, medications, latex, and bee venom, but can also occur in the absence of a specific allergenic trigger. Due to the nature of this medical condition, Epinephrine injectors designed for emergency use, must be available at all times. The injectors must be discarded when they expire or after use, which proves very wasteful since use is rare. It is advisable to carry two doses, for those at risk of anaphylaxis, as a second dose may be required in some instances. This effectively doubles the waste of one-time use injectors in accordance with the state of the art.

There are several drawbacks associated with such injector devices. For example, medications used in the emergency setting, such as Epinephrine are expensive. In addition, most injectors can only deploy a single dose of medication, and are very often discarded unused as the medication within the injector loses efficacy and reaches expiration typically in 12 to 18 months. Furthermore, these injectors are bulky and must be treated as medical waste which requires a sharps container.

The combination of the short expiration date, the lack of use over years (particularly for chronic conditions), and the high costs of these devices, patients are deterred from refilling this medication. As a result, they risk not having the proper treatment when needed.

Likewise, public venues such as restaurants, sporting arenas, amusement parks, schools and the like are also deterred from having injectors available as part of an emergency kits, in part because they are not required to carry them, and in part because it is inconvenient to keep them. Increasing availability in such public places would lead to quicker treatment and reduce reaction severity.

Epinephrine is also required in medical or dental offices, hospital clinics, and by emergency medical service (EMS) personnel. However, many do not carry Epinephrine and if they do, it is often drawn up from a vial into a syringe rather than being dispensed from an injector, since the high price and frequent need to refill is dissuasive. Having a ready-to-use injector in these settings can decrease the time to injection of Epinephrine and other medications used in the emergency setting.

Accordingly, there is a need in the art for cost effective, reusable auto-injector systems and methods as disclosed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide an injector device.

It is another aspect of the disclosed embodiments to provide systems and apparatuses for auto-injectors.

It is another aspect of the disclosed embodiments to provide methods and systems for reusable auto-injector devices with refillable medication cartridges and replaceable needles.

The disclosed embodiments provide auto-injectors for dispensation of emergency medications such as the epinephrine, but may have applications for other injectable medications as well. The embodiments incorporate both a replaceable needle and replaceable cartridges or vials containing medicament. Instead of having to replace the entire device every time following each use, or when the Epinephrine expires, users only have to replace the needle and cartridge containing medication, at a much lower cost to them. The lower cost may help make these types of medications more available in public venues as part of their first aid kits, and may help patients be more compliant with on-time refills. The ability to reuse the auto-injectors disclosed herein, will also impact the environment by creating significantly less medical waste, as only the expired cartridge needs to be discarded if the injector is unused.

It will be appreciated that the disclosed methods and systems can be achieved according to the embodiments herein. For example, in an embodiment, an injection system comprises a housing unit structured around loading tray configured to accept a medication cartridge, a needle situated below this loading tray to transport medication from the cartridge to the injection site, a sliding protective sheath surrounding the injection end of the housing, a plunger comprising at least two telescoping sections, built within the housing, which is capable of imparting a downward force on one end of the medication cartridge for the release of medication through the other end. In an embodiment, the needle can be further classified as a double-sided needle comprised of a first pointed end, a second pointed end, and a connective region capable of transporting medication between either end. In an embodiment, one of the first pointed end and the second pointed end of the two sided needle is configured to pierce the medication cartridge.

In an embodiment, the system further incorporates a sheath spring configured to extend and retract the protective sheath. In an embodiment, the injection further utilizes a non-electrically-actuated force generator positioned to impart a downward force on the plunger.

In an embodiment, a removable safety cap is configured to prevent the plunger from imparting any downward force on the medication cartridge.

In an embodiment the injection system further employs a needle hub configured to hold the needle component. The system further includes a needle switch operably connected to the needle hub capable of uniting the disposable needle to the sub-component

In an embodiment, the injection system further includes a cartridge insertion door connected to the overall housing unit that provides the user with access to the load tray.

In another embodiment an auto-injector comprises a medication cartridge, a housing unit with a loading tray configured to accept the medication cartridge, a double-sided needle configured to dispense medication from the medication cartridge, a sheath surrounding the injection end of the housing, built to be extended and retracted by a sheath spring, a telescoping plunger, a non-electric force generator, such as an injection spring, configured to impart a downward force on the plunger in order to transfer the force on the medication cartridge for dispensation of the medication from the opposite end of the cartridge into the needle.

In an embodiment, the needle can be further classified as a double-sided needle comprised of a first pointed end, a second pointed end, and a connective region capable of transporting medication between either end.

In an embodiment, the medication cartridge further utilizes a cap, or septum, capable of being pierced by the double-sided needle for transfer of medication into the needle body. In an embodiment, the auto-injector further comprises a removable safety cap configured to prevent the plunger from imparting any downward force on the medication cartridge.

In an embodiment, the sheath further incorporates a needle aperture. In an embodiment, the auto-injector is further constructed to restrict movement of the medication cartridge between at least two sliding positions, specifically: a first sliding stop at which medication is dispensed from the cartridge; and a second sliding stop where the cartridge can be loaded and unloaded.

In an embodiment, the injection system of further employs a needle hub configured to hold the needle component. In an embodiment the auto-injector uses a needle switch operably connected to the needle hub capable of uniting the disposable needle to the sub-component.

In another embodiment, a modular injector system comprises a dispenser assembly built from an outer housing unit, a telescoping plunger in this housing, a non-electric force generator, specifically an injection spring, configured to impart a downward force on the plunger, and a protective sheath connected to the telescoping plunger to control the initiation of the injection process; and a cartridge assembly comprising an inner housing unit built with a loading tray configured to accept a medication cartridge, a needle configured to dispense medication from a medication cartridge to the injection site.

In an embodiment, the dispenser assembly is configured to operate around the cartridge assembly and control its movement between the two sliding positions. The dispenser assembly further comprises a sheath surrounding the injection end of the housing unit, which is designed to activate injection when retracted and through which the needle of the cartridge assembly can move to inject the medication.

### BRIEF DESCRIPTION OF THE FIGURES

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1A depicts an elevation view of an injection system, in accordance with the disclosed embodiments;

FIG. 1B depicts a perspective view of an injection system, in accordance with the disclosed embodiments;

FIG. 1C depicts a housing of an injection system, in accordance with the disclosed embodiments;

FIG. 1D depicts a needle associated with an injection system, in accordance with the disclosed embodiments;

FIG. 1E depicts a cartridge and needle assembly associated with an injection system, in accordance with the disclosed embodiments;

FIG. 2 depicts an exemplary flow chart illustrating the use of an injection system, in accordance with the disclosed embodiments;

FIG. 3 depicts an exemplary flow chart illustrating the mechanism of operation of an injection system, in accordance with the disclosed embodiments;

FIG. 4A depicts aspects of a locking mechanism associated with an injection system, in accordance with the disclosed embodiments;

FIG. 4B depicts aspects of a plunger associated with an injection system, in accordance with the disclosed embodiments;

FIG. 4C depicts aspects of a telescoping plunger associated with an injection system, in accordance with the disclosed embodiments;

FIG. 4D depicts the relationship between a telescoping plunger and a platform associated with an injection system, in accordance with the disclosed embodiments;

FIG. 5 depicts another embodiment of an auto-injector system, in accordance with the disclosed embodiments;

FIG. 6A depicts aspects of a modular auto-injector system, in accordance with the disclosed embodiments;

FIG. 6B depicts aspects of a modular auto-injector system, in accordance with the disclosed embodiments;

FIG. 7A depicts aspects of a dual auto-injector system, in accordance with the disclosed embodiments;

FIG. 7B depicts aspects of a dual auto-injector system, in accordance with the disclosed embodiments;

FIG. 8A depicts aspects of an auto-injector system, in accordance with the disclosed embodiments;

FIG. 8B depicts aspects of an auto-injector system, in accordance with the disclosed embodiments;

FIG. 9 depicts aspects of a stacked auto-injector system, in accordance with the disclosed embodiments;

FIG. 10 depicts aspects of an auto-injector system, in accordance with the disclosed embodiments;

FIG. 11 depicts aspects of a gas driven auto-injector system, in accordance with the disclosed embodiments;

FIG. 12A depicts aspects of a trigger locking auto-injector system, in accordance with the disclosed embodiments;

FIG. 12B depicts deployment of a trigger locking auto-injector system, in accordance with the disclosed embodiments;

FIG. 13A depicts aspects of a simple modular auto-injector system, in accordance with the disclosed embodiments; and

FIG. 13B depicts additional aspects of a simple modular auto-injector system, in accordance with the disclosed embodiments.

### DETAILED DESCRIPTION

Embodiments and aspects of the disclosed technology are presented herein. The particular embodiments and configurations discussed in the following non-limiting examples can be varied, and are provided to illustrate one or more embodiments, and are not intended to limit the scope thereof.

Reference to the accompanying drawings, in which illustrative embodiments are shown are provided herein. The embodiments disclosed can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims. Aspects of the embodiments disclosed herein can be readily incorporated with other embodiments, without departing from the scope of the disclosure.

The embodiments disclosed herein are generally directed to reusable auto-injector devices with refillable medication cartridges and replaceable needles. The systems and methods allow the refillable cartridge to be inserted into the auto-injector. After medication is administered, the needle can be replaced, and a new medication cartridge can be installed. The auto-injectors can be easily reset for reuse with a new needle and new medication cartridge.

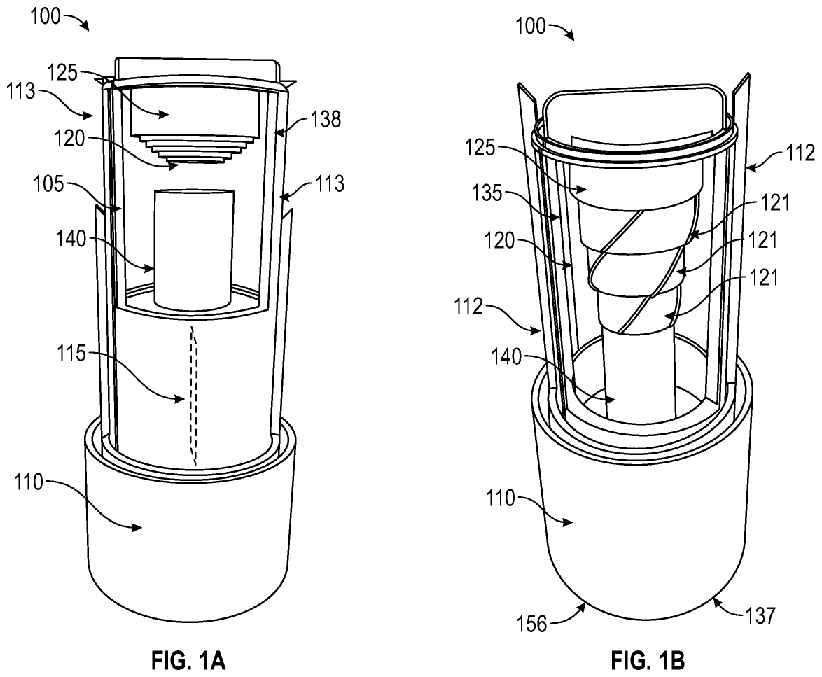

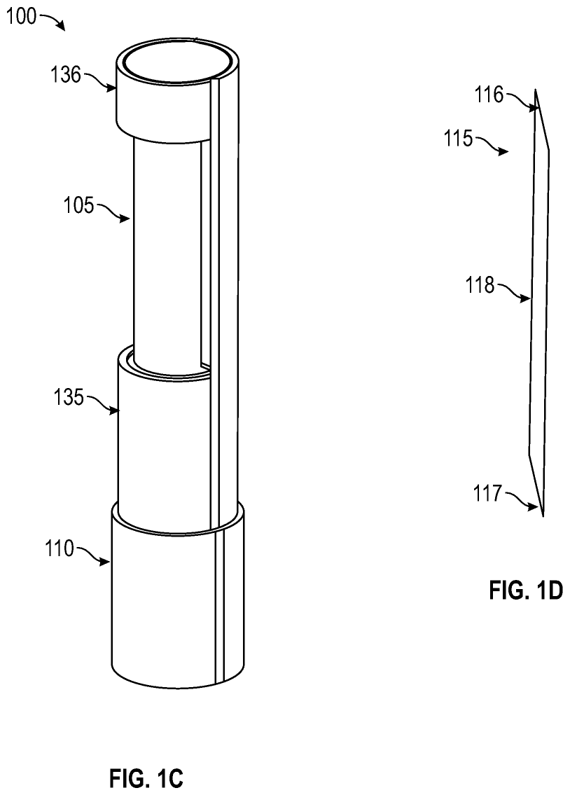

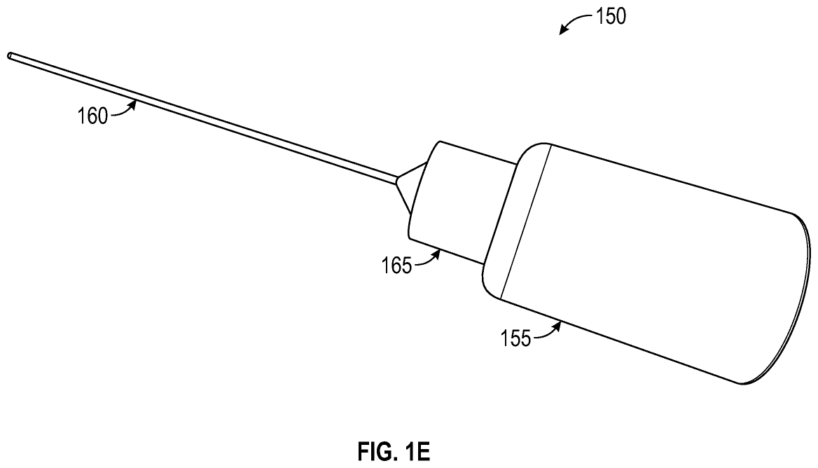

FIG. 1A-1E Illustrate aspects of an injection system 100 in accordance with the disclosed embodiments. The injection system 100 can be embodied as an auto-injector system 100 and can comprise subsystems including a cartridge loading assembly 105, a retractable protective sheath 110, a needle 115, and a telescoping plunger 120. The system 100 further includes a housing 135 for housing the subsystems. An external elevation view of the housing is provided in FIG. 1C. These sub-systems work in tandem to safely deliver medication from a medication cartridge 140 to a patient, while advantageously reducing the number steps in the injection process and the associated waste.

The system 100 can make use of a generator 125 configured to impart downward force on the plunger 120. In certain embodiments, the generator 125 can comprise an injection spring, but other force generating systems can also be used. For example, in certain embodiments, the generator can comprise an electrically powered linear actuator, pneumatic system, step motor, electromagnetic actuator, gas system, gear system, or the like. The plunger 120 can comprise telescoping sections 121, configured inside the housing 135 and in operable communication with the generator 125. The plunger 120 can impart a downward force on one end of the medication cartridge 140 for the release of medication through the needle 115. In certain embodiments, a sheath spring 130 and associated assembly further detailed herein, is configured to extend and retract the protective sheath 110.

In an exemplary embodiment, the housing 135 is structured around a loading tray associated with the cartridge loading assembly 105, which is configured to accept a medication cartridge 140. The needle 115 can be proximate to the loading tray. In an exemplary embodiment, the needle 115 is situated below the loading tray, and is configured to transport medication from the medication cartridge 140 to an injection site. The sliding protective sheath 110 surrounds the injection end of the housing 135 and prevents the needle 115 from being exposed until the injector system 100 is in use. This reduces the potential for accidental needle sticks.

FIG. 1A illustrates the plunger 120 and telescoping plunger sections 121 in a compressed state. In this configuration a second slide stop is illustrated in association with the cartridge 140. FIG. 1B illustrates the plunger 120 and associated plunger sections 121 in an uncompressed state. In this configuration the first slide stop is illustrated in association with the cartridge 140. In operation, the generator 125 can provide a downward force on the plunger 125 upon activation by a user. The plunger 125 can include telescoping sections 121 which extend downward to provide force on the medication cartridge 140. The downward force on the medication cartridge 140 pushes the medication cartridge 140 onto the needle 115. The needle 115 pierces the medication cartridge 140. Simultaneously, the protective sheath 110 slides up the housing 135 exposing one end of the needle 115, on the delivery side of the injector system 100, to dispense medication from the medication cartridge 140 to an injection site on the patient.

Aspects of the needle 115 are illustrated in FIG. 1D. The needle 115 can comprise a double-sided needle including a first pointed end 116, a second pointed end 117, and a connective region 118 capable of transporting medication between the respective ends. In certain embodiments, the first pointed end 116 or the second pointed end 117 of the two sided needle 115 is configured to pierce the medication cartridge. The other end serves as the patient delivery end, for delivering the mediation to the patient at the injection site.

FIG. 1E illustrates an alternative configuration comprising a cartridge and needle assembly 150. In such an embodiment, the cartridge and needle assembly 150 can replace the separate cartridge 140 and needle 115. As illustrated, the cartridge and needle assembly 150 comprises a cartridge 155 connected to a needle 160 via a needle cone conduit 165. The needle cone conduit 165 fluidically connects the cartridge 155 to the needle 160. The needle cone conduit 165 is used to connect the top of the current main body to the bottom of the needle 160, which extends to its final point at the other end. The cartridge and needle assembly 150 can be a single use device which can be loaded into the loading tray 106 of the loading assembly 105.

It should be appreciated that the needle 115 or the needle 160 can comprise a 22 gauge needle. In other embodiments, other needle sizes and gauges can be used according to the specific application.

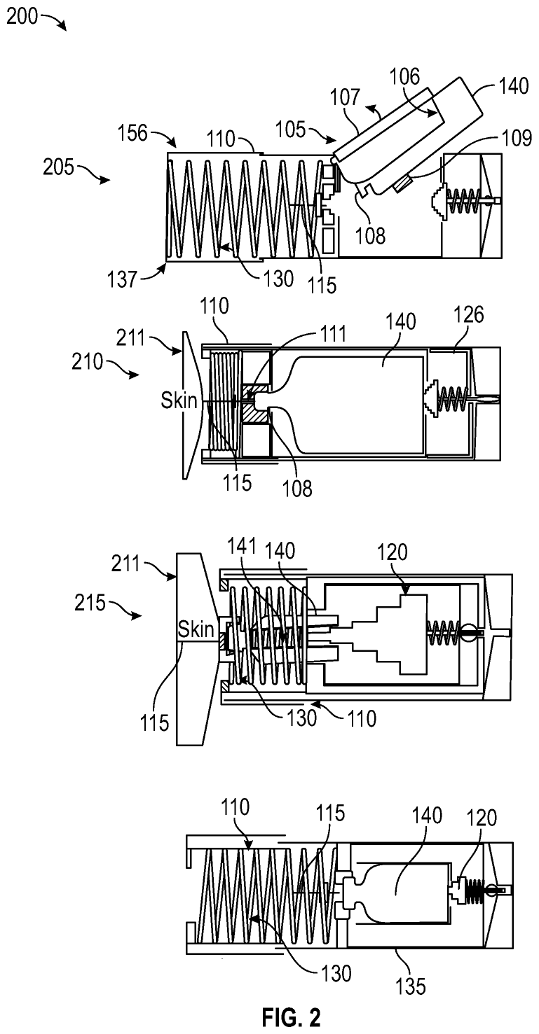

FIG. 2 illustrates additional aspects of the injection system 100, and steps associated with a method 200 for use of the systems disclosed herein. It should be appreciated that certain aspects of the system 100, as illustrated in FIG. 2, are disproportionately sized for illustrative purposes only, and other aspects are omitted in order to show how the system works.

As illustrated at step 205, a cartridge 140 (or other such vial containing medication) can be loaded into the loading tray 106 of the loading assembly 105. In certain embodiments, the tray 106 can be released with a button on the side of the auto-injector, or by pushing open a swinging door 107.

In an alternative embodiment, a removable panel can be pulled away from the housing 135 to expose the loading tray 106 for the cartridge 140. In another embodiment, a spare cartridge 140 and/or a spare needle can be housed within the system 100 so as to make it convenient and compact, especially in the case of epinephrine where the recommendation is to carry two doses at all times.

The cartridge 140 containing medicament is loaded into the loading tray 106, cap 108 side first. The cap 108 of the cartridge 140 can comprise a sealing material that can be penetrated by the needle 115 when the needle 115 contacts the cap 108. Once the cartridge is in place, the cartridge loading assembly 105 can be closed. The cartridge 140 can include a protrusion 109 so that the cartridge loading assembly 105 will not shut if the cartridge 140 not loaded properly. This is a safety feature to prevent improper loading.

The cartridge loading assembly 105 can also be configured to be transparent so that the cartridge 140 is visible when the cartridge loading assembly 105 is closed. In certain embodiments, the housing 135 can include a clear window 136 adjacent to the cartridge 140 that allows the user to view the contents and information on the cartridge 140, such as the name of the medication and the expiration or “use by” date.

After the cartridge 140 is loaded, a safety cap 137, located on the front end 156 of the injector system 100, can be removed or released. The safety cap 137 is used to prevent the injection spring 125 and plunger 120 from accidental activation. In order to initiate deployment of the medicament, at step 210, the retractable sheath 110 is pushed against the skin 211. The pushing force slides the sheath 110 along the body 135 and activates the plunger 110 as further detailed herein.

The generator 120 exerts a force on the plunger 125 pushing the medication cartridge 140 toward the delivery side of the housing. The cap 108 of cartridge 140 is pushed into the opening 111 formed in the housing 135, proximate to the needle 115 at the delivery side of the system 100. One end of the double-sided needle 115 penetrates the cap 108 of the cartridge 140.

At step 215, the delivery side of the needle 115 can be exposed as the protective sheath 110 slides back along the housing 135. The needle 115 is pushed into the injection side on the patient at the injection site. The medicament 141 from the cartridge 140 can flow through the needle 115 into the patient. The auto-injector system 100 can be held in position for a few seconds to ensure that all of the medication 141 has been released from the cartridge 140.

At step 220, the injector system 100 can be pulled away from the injection site after the injection is complete. As the system 100 is pulled away, the protective sheath 110 slides back down the housing 135, while the cartridge 140 and needle 115 are retracted back into the housing 135. This deactivates the plunger 120. The used cartridge 140 and/or needle 115 can then be removed from the auto-injector system 100. A new cartridge 140 and/or needle 115 can then be loaded for future use.

A safety feature of the auto-injector system 100 is that the needle 115 can be replaced. The housing 135 and sliding sheath 110 prevent unwanted exposure to the needle, and protect the patient from accidental injection.

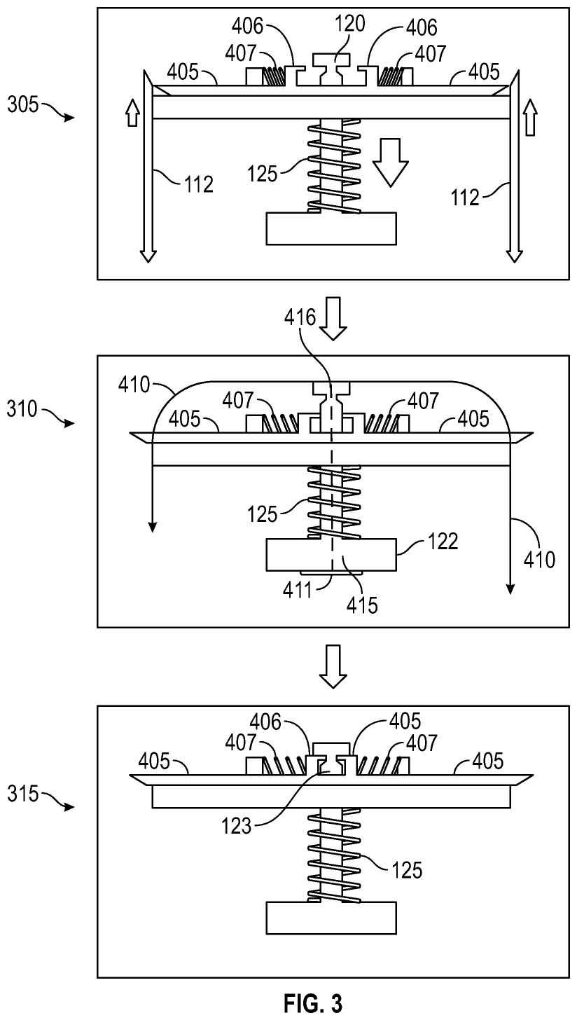

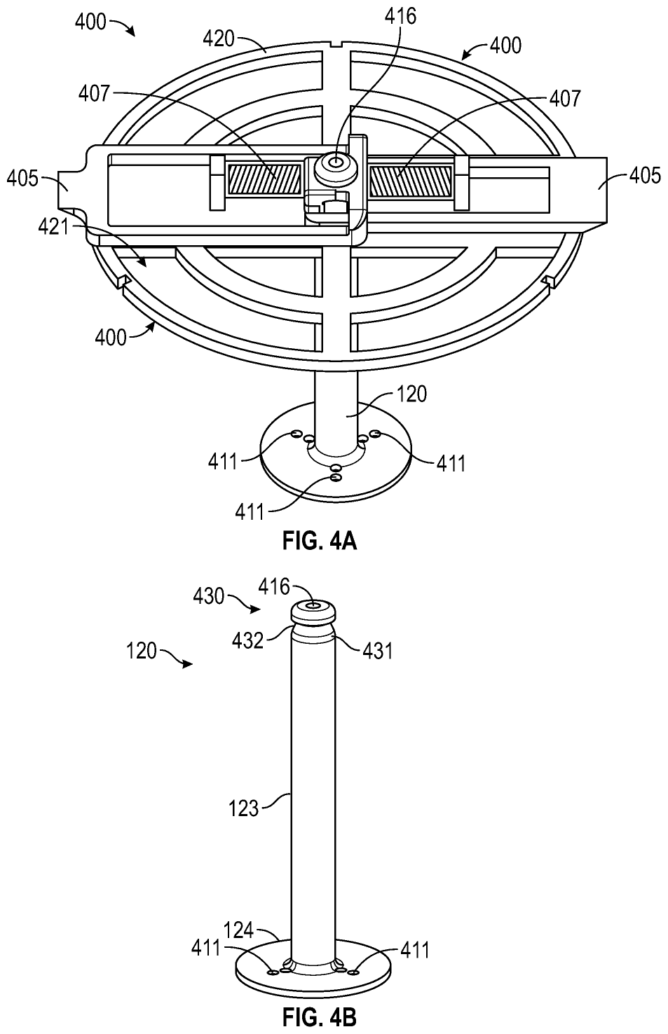

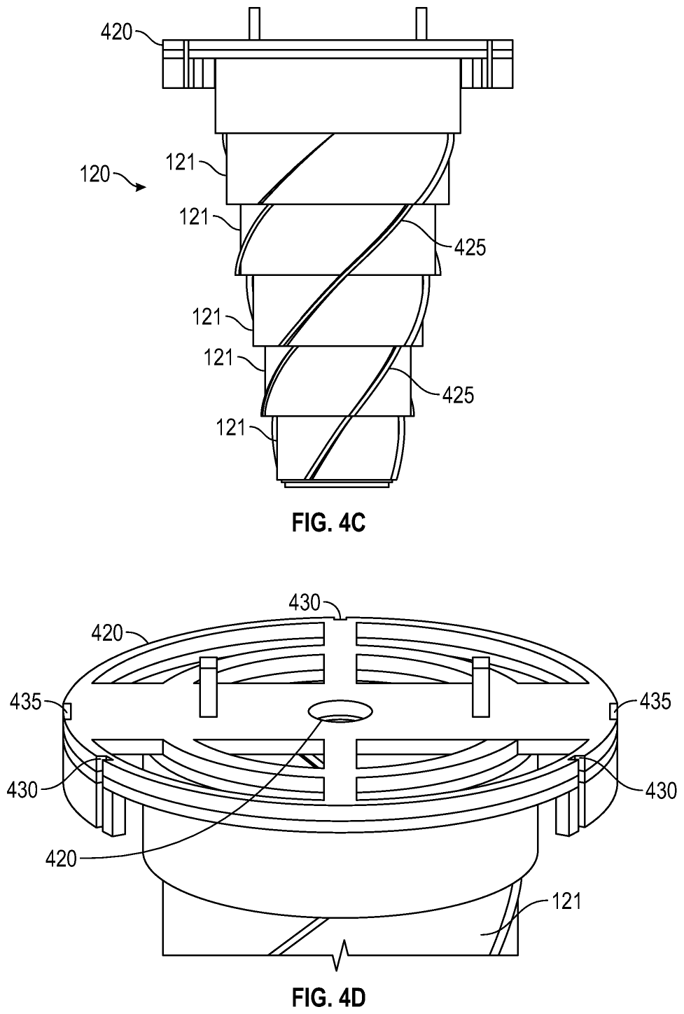

An aspect of the disclosed embodiments is that the protective sheath 110 slides up the housing body, while the needle and cartridge are forced to the delivery end of the system, exposing the needle for injection. In certain embodiments, the normal force applied to the sheath 110 by pressing it against the injections site, activates the generator 125 and or release the plunger 120 to push the cartridge 140 into the needle and the needle into the patient. FIG. 3 illustrates the process associated with these coordinated movements and FIG. 4A-4D Illustrates additional aspects of the system used to accomplish the coordinated movement.

Step 305 of FIG. 3 illustrates the deployment of the plunger 120. When the protective sheath 110 is applied to the injection point, the normal force against the skin pushes the sheath along the housing 135. Retractable sheath bars 112, slide along bar tracks 113 formed in the walls 138 of the housing 135. As the retractable sheath bars 112 reach their uppermost position, they can make contact with sliding arms 405.

The sliding arms 405 are configured with plunger clips 406 configured to engage the plunger 120. The plunger clips 406 can be biased against the plunger 120 with plunger clip springs 407. The retractable sheath bars 112 press the sliding arms 405 inward. This inward motion compresses the plunger clip springs 407 and disengages the plunger clips 406 from the plunger 120, opening the path through the housing 135 for the plunger to move downward. It should be appreciated that in FIG. 3, the generator 125, which imparts the downward force on the plunger 120, is illustrated as a spring, but in other embodiments, other force generating systems can be used.

Step 310 of FIG. 3 illustrates the retraction of the plunger 120, after the injection has been delivered. As the system 100 is pulled away from the injection site, the sheath spring 130 forces the protective sheath 110 to slide back along the housing into an extended position. This disengages the retractable sheath bars 112 from the sliding arms 405.

Control wire 410 can be connected on one end to the retractable sheath 110, and can run through a channel 415 in the plunger 110 where they connect to the plunger face 122 at connection point 411. In certain embodiments, the control wire 410 can comprise a cable of multiple (e.g., two or more) control wires 410 which are intertwined as a cable in the plunger, and then separated into separate wires as they exit the channel exit 416, with each wire 410 connecting to a different point on the protective sheath 110.

The control wires 410 connected to the protective sheath use the motion of the sheath to pull the plunger 120 back against the plunger spring 125, retracting the plunger. The control wires can extend along the tracks 113 formed in the walls 138 of the housing 135.

Step 315 illustrates the system 100 at reset for further use. When the retractable sheath 110 reaches its fully extended position, the control wires 410 (not shown at step 315) apply less force on the front 122 of the plunger 120. At this stage, the force exerted by the plunger spring 125 reaches a balance with the sheath spring 130, with the front of the plunger 122 in a locked position. The locked position is secured by the sliding arms which are engaged with the plunger shaft 123 via the plunger clips 406 and plunger springs 407.

Various details of the auto-injector system 100 are illustrated in FIG. 4A-4D. FIG. 4A illustrates aspects of the locking assembly 400 in accordance with the disclosed embodiments. The locking assembly can include the sliding arms 405 and springs 407. The sliding arms can be mounted on a rigid platform 420, which can, in certain embodiments, include a series of interconnected concentric circles 421. The plunger 120 can extend through the center most concentric circle 421 where it is engaged with the sliding arms 405.

FIG. 4B illustrates aspects of the plunger 120 in accordance with the disclosed embodiments. The plunger 120 can comprise a shaft 123 with a base 124. The base 124 includes connection points 411 for control wires 410. The channel exit 416 can be formed on the head 430 of the plunger 120. The head 430 further includes a tapered neck 431 and lip 432 configured to engage with sliding arms 405.

FIG. 4C illustrates aspects of the telescoping plunger sections 121 in accordance with the disclosed embodiments. In certain embodiments, the telescoping sections 121 can include helical guides 425 configured to allow each of the respective telescoping sections 121 to rotate into or out of a nested position with the telescoping section 121 above or below it. The plunger can be connected to the platform 420. FIG. 4D illustrates a perspective view of the telescoping plunger 120, showing the centermost concentric circle 421 through which the plunger 120 extends. The platform 420 can include wire track cutouts 430 which serve as wire guides for the control wires 410. The platform also includes retractable sheath bar guides 435 configured to accept the retractable sheath bars 112.

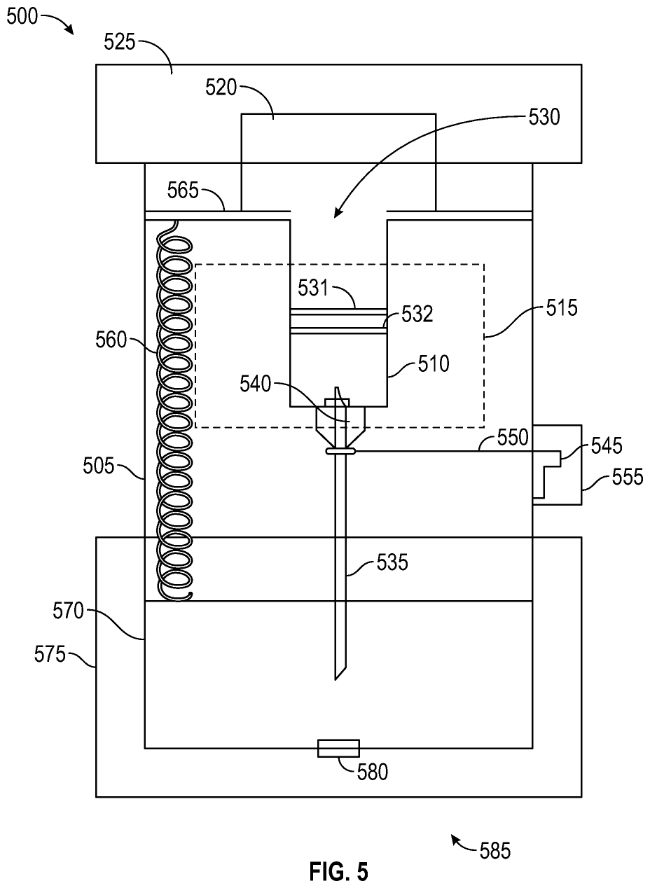

FIG. 5 illustrates a diagram of an embodiment of an auto-injector system 500 in accordance with the disclosed embodiments. It should be appreciated that the aspects of this and other embodiments can be used with one another. The auto-injector system 500 comprises a housing 505 configured to hold a medicament cartridge 510. An insertion door 515 is formed in the housing and allows the cartridge 510 to be inserted in the housing. In this embodiment, a plunger button 520, protected by a safety cap 525 can be used to depress a plunger 530. The plunger includes a first slide stop 531 and a second slide stop 532. When the plunger 530 is depressed the cartridge 510 is pressed against a needle 535. In certain embodiments, the needle 535 comprises a double sided needle. The needle 535 is secured in a needle hub 540. The needle hub is connected to a needle switch 545 with a connector 550. The needle switch 545 is protected by a safety cap 555.

Spring 560 is attached on one end to slider 565 and on the other end to retractable sheath 570. The retractable sheath 570 comprises an enclosure for the needle 535, with a needle opening 580 at the delivery end 585 of the injector. The sheath is covered by bottom safety cap 575.

The cartridge 510 is replaceable through the door 515, integrated in the housing 505. This door 515 allows the user to remove the expired cartridge from and insert a new cartridge. The two sliding stops 531 and 532 prevent the medication from spilling when the cartridge 510 is replaced. Once the cartridge 510 is properly installed, the needle 535 can be inserted into the cartridge 510. In this embodiment, the needle insertion should only be executed when the user is ready to administer the injection. To insert the needle 535 into the cartridge 510 the safety cap 555 on the right-side of the housing 505 can be lifted, and the switch 545 is turned upward. This process lifts the connector 550 upward, which in turn inserts the needle 535 upward into the cartridge 510.

After the needle 535 is inserted, the safety cap 555 is lowered to ensure it is not flipped downward before injection. Once the needle 535 is inserted into the cartridge 510 and the safety cap 555 is returned to the downright position, the auto-injector system 500 is ready to administer an injection. First, the top safety cap 525 and bottom safety cap 575 are removed. Next the retractable sheath 570 can be depressed onto the injection site to expose the needle. The plunger button 520 can then be operated. The plunger button 520 is attached to a plunger 530 and a slider 565. When the button 520 is pushed the slider 565 compresses the spring 560. The spring motion and the force on the button 520 allows the medication to enter the double-ended needle 535 and move through to the end of the needle 535 into the injection site.

Once injection is complete, the needle 535 is retracted from the injection site, and the retractable sheath 570 extends back out to protect the needle. The safety cap 525 and safety cap 575 can be replaced for added safety.

In certain embodiments the system 500 can be divided into subsystems. One subsystem contains the top safety cap to the medication cartridge. The other subsystem contains the outer housing, plunger, plunger button, and bottom safety cap. The first subsystem is a replaceable unit that extends from the top safety cap to the cartridge. The second subsystem is a reusable unit that houses the plunger and plunger button. Instead of carrying two whole devices, the user can carry two of first subsystem and one of second subsystem. This decreases the bulkiness of the device and encourages patients to have their device with them at all times.

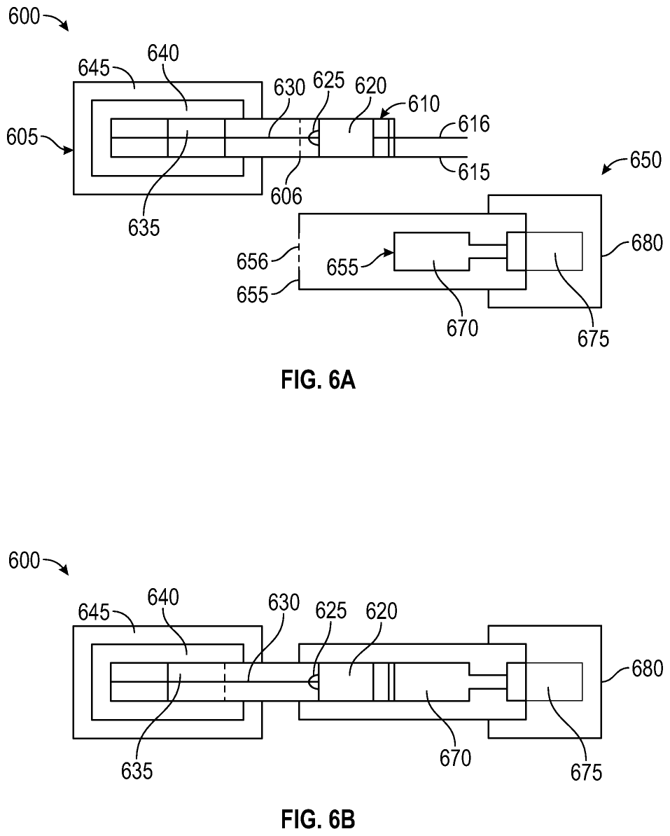

FIG. 6A and FIG. 6B illustrate aspects of a modular auto-injector system 600. It should be understood that the modular auto-injector system 600 can incorporate aspects of other embodiments, without departing from the scope disclosed herein.

The modular auto-injector system 600, includes a delivery assembly 605 and a dispenser assembly 655. The delivery assembly 605 can be connected to the dispenser assembly 655 by engaging male threads 606 on the housing 610 of the delivery assembly 605 to female threads 656 on the housing 660 of the dispenser assembly 655. FIG. 6A illustrates the delivery assembly 605 separate from the dispenser assembly 655. FIG. 6B illustrates the delivery assembly 605 connected to the dispenser assembly 655.

The delivery assembly comprise a first slide stop 615 and second slide stop 616 configured to engage a cartridge 620. A needle hub 625 holds a needle 630. The needle extends through a needle holder 635 inside a retractable sheath 640, covered by a safety cap 645. The dispenser assembly 655 can comprise a plunger 670, connected to a plunger button 675 and a safety cap 680.

Once the delivery assembly 605 is connected to the medication assembly 655, the end of the cartridge 620 lines up to the free end 671 of the plunger 670. The safety cap 680 and safety cap 645 can be removed. Next the retractable sheath 640 can be positioned at the injection site. The force on the retractable sheath exposes the needle 630 which can penetrate the injection site. Once the needle 630 is inserted the plunger button 675 can be depressed. The plunger button 675 pushes the plunger 670 into the cartridge 620, releasing the medication through the needle 630 into the injection site.

Once injection is completed the needle 630 is pulled out of the injection site, and the retractable sheath 640 extends to cover the needle 630. The safety cap 645 and safety cap 680 can be replaced. After returning the safety caps are replaced the dispenser assembly 655 can be unscrewed from the delivery assembly 605. In certain embodiments, the delivery assembly 605 can be discarded. The dispenser assembly 650 can be retained for future used and can be reset by pushing the free end 671 of the plunger 670 to its original position.

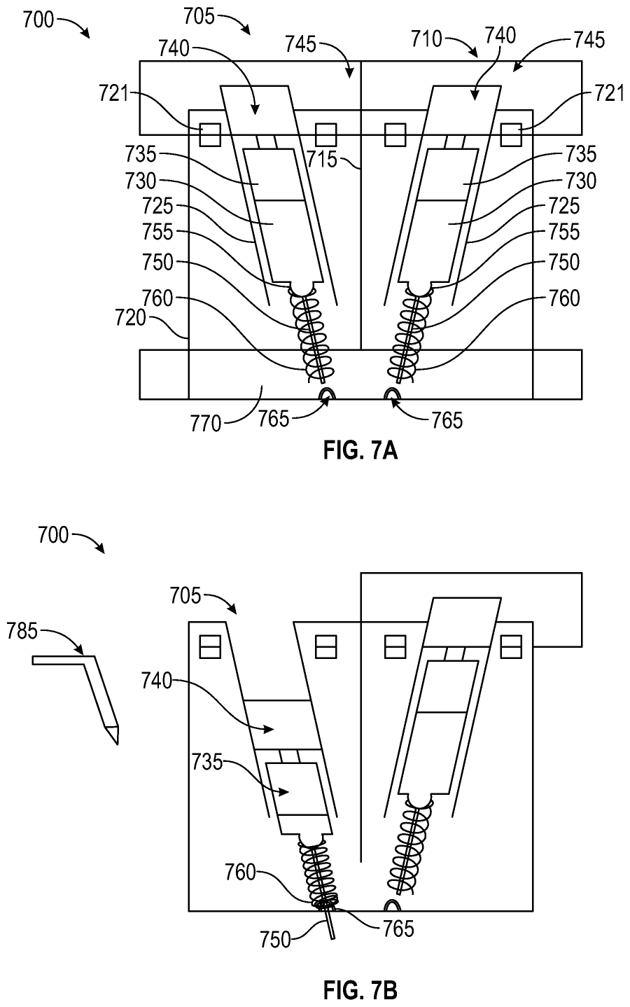

FIGS. 7A-7C illustrate a dual auto-injector 700, in accordance with another embodiment that carries two doses of medicament. The dual auto-injector comprises two injector assemblies, assembly 705 and assembly 710. Each of the injector assemblies has identical but mirrored components. The assembly 705 and assembly 710 are connected at interface 715. Each of the respective assemblies comprise a housing 720 with an attachment clasp 721 configured to attach the respective assembles. The housing 720 includes a sheath 725 configured to hold a cartridge 730. A plunger 735 is operably connected to a plunger button 740 with a safety cap 745. A needle 750 is connected to the cartridge 730 with a needle hub 755. A needle spring 760 surrounds the needle 750, which is aligned to the needle opening 765. The needle opening 765 is covered by a safety cap 770. Each of the respective assembly 705 and assembly 710 can be designed at an angle so that the injection openings 765 are as centered as possible.

To assemble the system 700, the needle safety cap 770 can be removed. The needle end slides into the open slot in the device. Once positioned, the top safety cap 745 can be pressed down until the attachment 721 clasps on the bottom of assembly 705 or 710 respectively attach to the attachment clasps on the top of the system 700.

To administer an injection as illustrated in FIG. 7B, one of the top safety caps 745 can be removed, and the bottom safety cap 770 can be removed. The bottom of the auto-injector assembly 705 can be position at the injection site. The plunger button 740 can be depressed which pushes the medicament 735 through the needle 750 into the injection site. The plunger button 740 force also activates the spring 760 on the needle 750 which moves the entire plunger system forward, resulting in the needle extending through the hole 765 and into the injection site. Once the injection has been completed the spring 760 will extend and return the needle 750 back into the housing 720.

To remove the replaceable assembly 705 or 710, the user can insert the L-shaped “screw-driver” tool 785 that can be provided with the dual auto-injector 700. The point of the tool 785 can be inserted into the plunger button. The tool 785 can be pushed down into the plunger button 740 until the handle is slightly above the opening of the housing 720. The tool 785 can then be used to remove the assembly 705. Once removed, the needle safety cap can be replaced and the assembly 705 can be discarded.

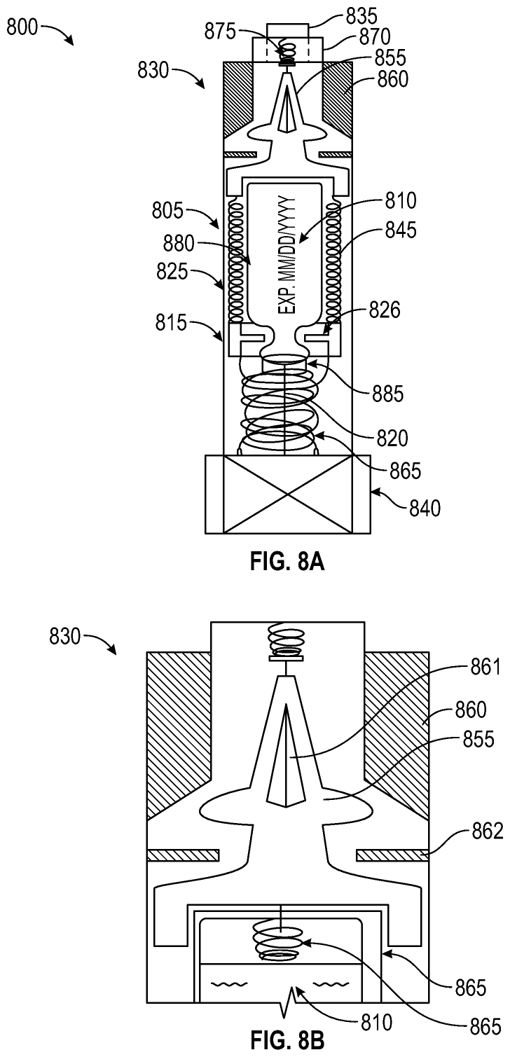

FIG. 8A illustrates an embodiment comprising a single-use auto-injector system 800. The system 800 includes a loading port 805 where the cartridge 810 is loaded and unloaded. The system further comprises a spring-loaded needle and plunger assembly 815 that, when activated, automatically inserts a needle 820 into the cartridge 810 and injects medicament into the patient.

The auto-injector system includes a body 825 with a lock assembly 830, locking in place the release button 835. The system also includes an injection safety cap 840 on the delivery side of the system 800.

The interior of the auto-injector system 800 is held in equilibrium by two sets of springs. The first set of springs are cartridge springs 845 configured at either side of the cartridge 810. The cartridge springs 845 are held in tension most of the time, as the top is locked in place by the injector body 825 and at the bottom by the cartridge lock 826. At injection, the cartridge springs 845 are necessary for the auto-insertion of the needle 820 and release of the medicament from the cartridge 810. The cartridge springs 845 are held in tension because they are locked in place.

The top locking assembly 830 is illustrated in FIG. 8B. The brace 855 is locked in position by the injector body holding it up. The brace can be compressible, so, when pushed against the chamfers 860 above it, it compresses to a point where it can slip through the slot 865 below and the cartridge springs 845 compress into place. The top locking assembly includes the central rod 861, and sliding lock 862.

The second set of springs are needle springs 865, which can be wrapped around the needle 820. These needle springs 865 maintain the regular position of the cartridge springs 845 by pushing them back into their locked position after injection; however, during injection, the locked position becomes impossible to reach, so their effect is nullified.

In order to activate the auto-injector system 800, the safety caps can be removed. When the cap is removed, both the release button 835 and a rotating knob 870 that is built around it for compactness. The button 835 includes a button spring 875. The button 835 is inaccessible at this point as it is locked in place below the top of the knob 870. This is one of the button's two conformational. Additionally, when the cap is removed, the loading port of the auto-injector system 800 can rotate away from the main body 825 to reveal a port 880 for the cartridge 810 to be inserted. The cartridge 810 is placed into this port 880, and the port 880 can then be rotated into place, flat against the body 825 of the auto-injector system 800. As the port 880 is rotated into place, the cartridge 810 is locked inside by arms that rotate out from the walls.

The knob 870 can then be rotated. With this knob's rotation, a sleeve extends itself from the back side of the auto-injector system 800 over the loading port 880 to prevent any unintentional motion of the cartridge-containing component. On top of rotating the sleeve, an O-ring 885 is lifted up and sealed against the outlet of the cartridge 810, and the needle 820 is inserted into the cartridge 810, exposing the needle 820 to the interior of the cartridge 810.

The medication can be injected by pressing the system 800 against the injection site and clicking the release button 835. Pressing the auto-injector system 800 against the injection site will raise the safety 840 that rests around the outside of the needle end of the auto-injector body 825. The safety 840 slides up the outside of the housing 825 while the needle 820 extends to penetrate the injection site. The needle spring 865, which wraps around the needle 820, is completely compressed in between the bottom of the cartridge 810 and the top of the safety 840, and the cartridge 810 is pushed back against the top of the needle 820. The compressible part of the cartridge can be pinched enough to slide through the slot below. When this happens, the cartridge springs 845 push both the cartridge 810 and needle 820 toward the injection site.

When the safety is pushed into place, a central, thin rod 861 pushes the release button 835 out of the locked position, extending a spring 875 at the bottom of the release button 835. The spring 835 holds the system 800 in this second conformational position. With the release button 835 unlocked, the button 835 can be pressed, pushing the thin rod 861 into the auto-injector body 825 and into the of the cartridge 810. The rod 861 inserts itself into a button at the bottom of the cartridge 810 that releases a spring inside the cartridge 810. The cartridges 810 can come pre-loaded with this spring inside in the locked position, but, when the spring is unlocked by the rod 861, the spring pushes a plunger to release the medicament through the needle 820 to complete the injection.

Rotating the knob 870 back will pull the sleeve away and the cartridge loading port will extend away from the auto-injector body once again to allow unloading of the used cartridge 810. The used needle can be replaced between uses. To do this, the retractable needle safety is pulled back until it clicks into place. At this point, the retractable safety 840 can be rotated until it tightens to a locked position and the needle 820 extends out the front end of the auto-injector system 800. From this conformation position, the needle 820 only needs to be unscrewed from its housing and the new needle put in its place.

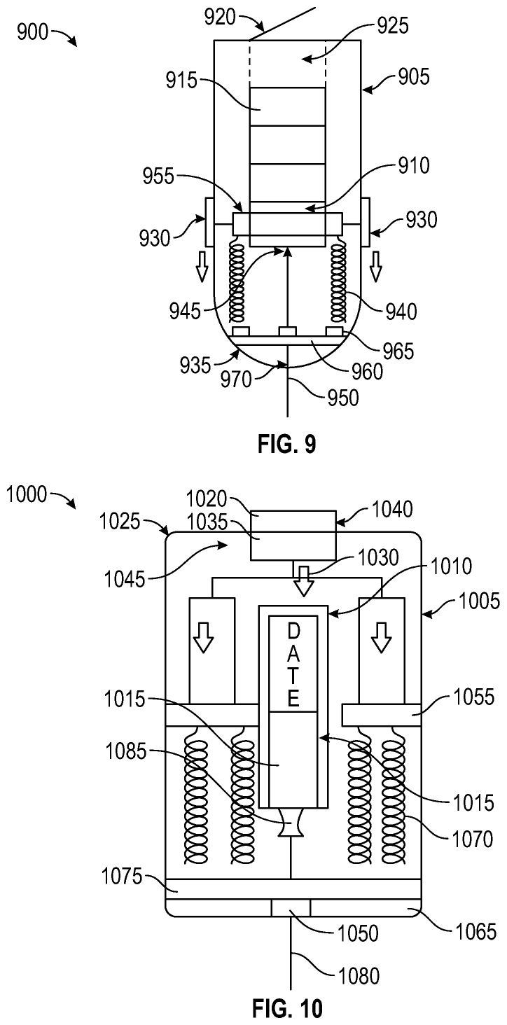

FIG. 9 illustrates a single use auto-injector system 900, which can be configured to allow unused medication cartridges to be removed after their expiration. The system 900 can include a housing 905 with gripping grooves and a window 910 in the housing to view information on the cartridges 915 such as expiration information. To replace the cartridge, a lid 920 on the back end can be opened. Old cartridge(s) 915 can be removed and new cartridge(s) 915 can be inserted. The new cartridges 915 are positioned in the correct location by tracks 930 located on either side of the cartridge opening 925.

The system 900 includes two handles 955 on either side of the housing 905, which can be pulled down towards the safety cap 935. By pulling the handles 955, the cartridge 915 closest to the safety cap 935 is engaged. As the handles 955 are pulled down, springs 940 on either side shorten, bringing the cartridge 915 closer to the medicament dispenser 945. The compressed spring 940 moves cartridges 915 via the handle 955 that connects the cartridges 915 on the track 930. Once the handles 955 are pulled down to most extended location, the medicament dispenser 945 will pierce the cartridge 915 and provide a route for the medication to be introduced to the needle 950.

After the cartridge 915 is engaged, the safety cap 935 can be pressed against the injection site for administration of the medication. The downward force against the injection site unsheathers the needle 950 by retracting the portion covered by the safety cap 935. This retraction is possible by the needle unsheather portion 960. The bottom of the device (safety lid side) can slide upwards towards the cartridge opening, occupying the space the needle unsheather 960 previously occupied, exposing the needle 950 through the needle stopper 970. In certain embodiments, sensors 965 are configured along the walls to also react to the force produced by pressing the system 900 on the injection site, and further compress the spring 940, aiding in the ejection of the needle.

FIG. 10 illustrates another embodiment of a single-use auto-injector system 1000 in accordance with the disclosed embodiments. The embodiment illustrated in FIG. 10 is intended to provide an easy to use user interface. The system 1000 includes a housing 1005 with a lid 1010. The lid 1010 provides access to allow cartridges 1015 to be loaded. The lid 1010 opens like a door and is wide enough to accommodate at least two cartridges 1015 containing medicament.

The system 1000 includes a locking mechanism 1020 that rotates around the top 1025 cross sectional area of the system 1000. The system 1000 includes a pusher 1030. The active portion 1035 only occupies about 20% of the total cross-sectional area to ensure safety. The active portion 1035 can be the same size as the locking mechanism flap 1040. Once the locking mechanism flap 1040 is rotated so that it aligns with the active portion 1035, the locking mechanism flap 1040 can be pushed down to activate the system 1000. The locking mechanism flap 1040 is kept on the inactive portion 1045 the device is not in use.

Before administration of medicament, the locking mechanism flap 1040 can be aligned with the active portion 1035. Once aligned the interface 1065 can be pressed against the injection site so that the the needle opening 1050 makes contact with the skin. The interface 1065 can be rubbery and can be flat to allow better traction to both skin and clothes. The needle unsheather 1075 is formed above the interface 1065. The downward pressing motion activates the needle system by providing a downward motion of springs 1070 and the spring contraction mechanism 1055 configured on the wall 1060 to unsheathe the needle 1080, and extend it out the needle opening 1050. The medicant dispenser 1085 provides mediation from the cartridge 1015 to the needle 1080, into the injection site.

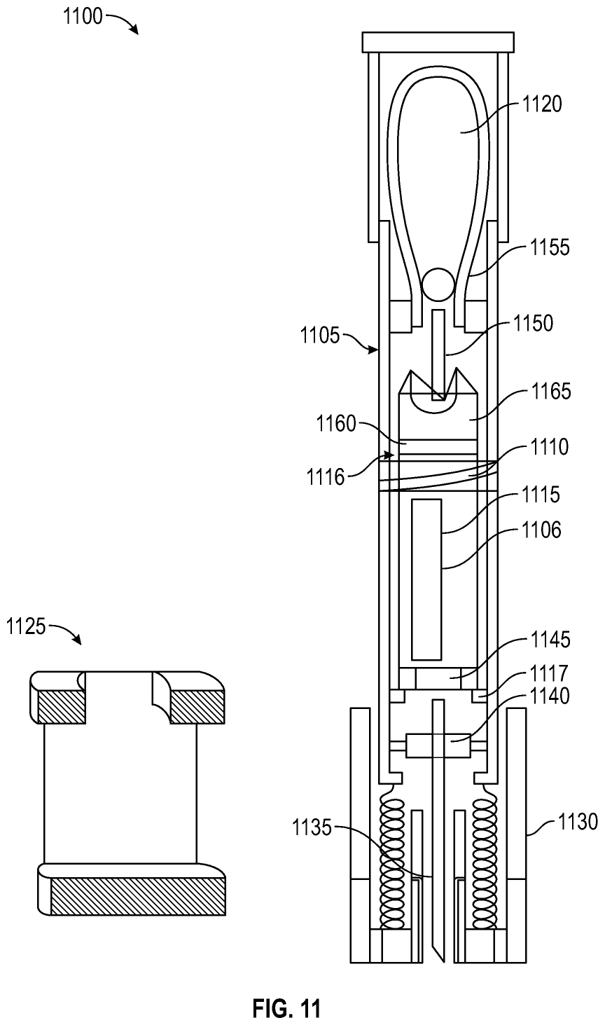

FIG. 11 illustrates a two-step auto-injector system 1100 in accordance with the disclosed embodiments. The system 1100 generally comprises a housing 1105 configured with a female threading 1110, configured to engage with male threading 1116 on a medication cartridge 1115. The cartridge is held in place with cartridge holder 1117

The housing 1105 can include a clear window 1106 to provide visual access to information printed on the cartridge 1115. A CO2 capsule 1120 is used to provide pressure to the medicant in the cartridge 1115. The CO2 capture 1120 can be replaceable. A safety cap 1125, prevents the retractable head 1130 from moving and also keeps the needle 1135 sterile and safe when the system 1100 is not in use. The top portion of the safety cap 1125 wraps around the housing 1105 above the retractable head 1130, and the bottom portion covers the retractable head 1130 and the needle 1135 inside. The safety cap 1125 does not encapsulate the entire device so that it is easy to remove with one hand.

When the two-step auto-injector system 1100 is pushed against the injection site, the retractable head 1130 retracts, unsheathing the needle 1135, while also pushing the stopper 1140 of the double-ended needle 1135 through the seal 1145 of the cartridge 1115. The stopper 1140 pushes the cartridge 1115 towards the back of the injector. As the cartridge moves back, the end forms a seal around the mouth of the CO2 capsule 1120. A needle-like piece 1150 on the end of the cartridge 1115 pushes the ball 1155 in the CO2 capsule 1120 back, releasing the CO2 into the chamber 1165 between the mouth of the capsule 1120 and the plunger 1160 inside of the cartridge. As the CO2 escapes the capsule 1120, this chamber 1165 expands, pushing the plunger 1160 towards the injection site, and expelling the medicament through the needle into the tissue.

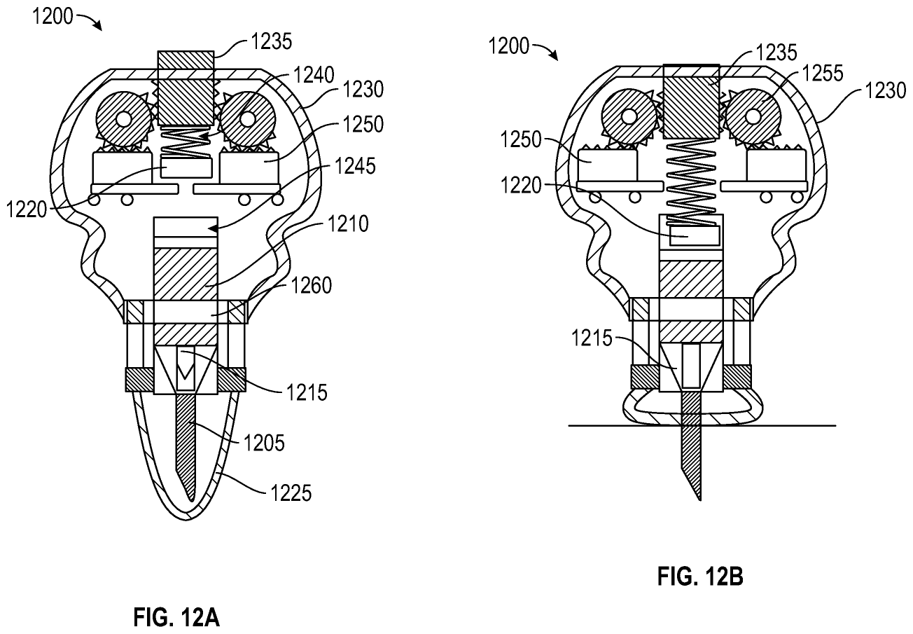

FIGS. 12A and 12B illustrate another embodiment of an auto-injector 1200, that utilizes a needle 1205 attached to a cartridge 1210. A valve 1215 at the base of the needle 1205 protects the medication from leaking, and only opens when pressure from the plunger 1220 pushes the medicament through the valve 1215.

The system 1200 includes a needle cap 1225 comprising a piece of rubber that retracts when it is pushed against the body, exposing the needle 1205. The needle 1205 can comprise a tear-free needle, which does not leave a hole in the cap 1225 once it has been extracted from it. The rubber cap 1225 can be reused, but can also be replaced by unscrewing the tip and placing a new one onto the injector housing 1230.

Once the needle 1205 is positioned against the injection site, a trigger button 1235 is used to release a spring 1240 inside which pushes down the plunger 1245 inside of the cartridge 1210.

FIG. 12B depicts the auto-injector system 1200 before and during use, showing how the trigger button 1235 moves the latching triggers 1250 out of the way of the plunger 1245 using a set of gears 1255. Once the plunger 1245 has been triggered, the pressure of the fluid within the cartridge 1220 makes the valve 1215 at the base of the needle 1205 open and release the medicament.

Once the auto-injector system 1200 has been used, it can be reset to its original configuration by first returning the triggers and plunger back to their locked positions. This can be accomplished by pushing the plunger 1245 back and locking it in place the triggers. The cartridge 1210 can be replaced by unscrewing the tip portion from the main housing 1230, unscrewing the cartridge 1210 from the tip, replacing it, then replacing the tip with the cartridge 1210 by screwing it into the housing 1230.

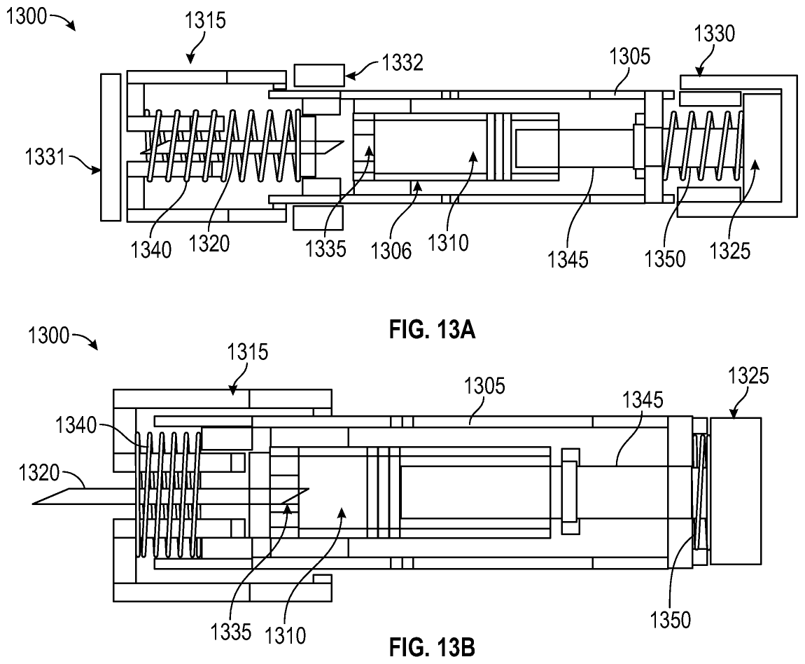

FIGS. 13A and 13B illustrate another embodiment of an auto-injector system 1300 that can be taken apart with a twisting mechanism in order to access the cartridge 1310 for replacement. The system 1300 includes a housing 1305 with a window 1306 which provides visual access to information printed on the cartridge 1310.

The system 1300 comprises a retractable head 1315, double sided needle 1320, medicament cartridge 1310, and plunger button 1325, as well as a button safety cap 1330 dispenser safety cap 1331, and sheath safety 1332.

FIG. 13A illustrates the system 1300 before use. The first step to use the system 1300 is to remove the safety caps. Next, the retractable head 1315 can be pressed against the injection site. Pressing the retractable head 1315 into the injection site retracts the retractable head 1315, as illustrated in FIG. 13B. When the system is not in use, the retractable head 1315 protects against needle exposure. The retractable head 1315 is biased to this position with head spring 1340.

During an injection, the head 1315 slides back towards the distal end of the housing 1305, pushing the double-ended needle through the seal 1335 in the cartridge 1310. Pushing the plunger button 1325 forces the plunger 1345 into the cartridge 1310 expelling the medicant through the needle 1320.

The system 1300 is advantageous because it has a simple mechanism for exchanging cartridges 1310, and the design minimizes the number of complicated, moving parts. The safety caps prevent the retractable head 1315 and plunger 1345 from moving before use, and ensure that the end of the needle 1320 is not exposed before use. There is a plunger spring 1350 under compression between the plunger button 1325 and the housing 1305 which keeps the button 1325 from inadvertently pushing out the medicant when the safety caps are off. The retractable head 1315 serves to keep the needle 1320 covered when the injector system 1300 is not in use.

The embodiments disclosed herein are directed to an auto-injector device which can be used with emergency medications such as the epinephrine. In other embodiments, the embodiments provided herein can be used with other applications for other injectable medications. The embodiments generally incorporate a replaceable needle and replaceable cartridges or vials containing medicament. The embodiments allow patients or providers to conveniently replace the needle and cartridge containing medication. The lower cost makes these types of medications more available in public venues, as part of first aid kits, and may facilitate timely medication refills. The ability to reuse the auto-injector will also impact the environment by creating significantly less medical waste, as only the expired cartridge needs to be discarded if the injector is unused.

Based on the foregoing, it can be appreciated that a number of embodiments, preferred and alternative, are disclosed herein. In an embodiment, an injection system comprises a housing with a loading tray configured to accept a medication cartridge, a needle configured to transport medication from the medication cartridge to an injection site, a sheath formed on an injection end of the housing, and a plunger comprising at least two telescoping sections wherein the plunger is configured to impart downward force on the medication cartridge in order to release medication in the medication cartridge.

In an embodiment, the needle further comprises a two sided needle comprising: a first pointed end, a second pointed end, and a body connecting the first pointed end and second pointed end. In an embodiment, one of the first pointed end and the second pointed end of the two sided needle is configured to pierce the medication cartridge.

In an embodiment, the injection system further comprises a sheath spring configured to extend and retract the sheath.

In an embodiment, the injection system further comprises a generator configured to impart a downward force on the plunger.

In an embodiment, the injection system further comprises a removable safety cap configured to prevent the plunger from imparting downward force on the medication cartridge.

In an embodiment, the injection system further comprises a needle hub configured to hold the needle. In an embodiment, the injection system further comprises a needle switch operably connected to the needle hub wherein the needle hub holds the needle in alignment with the medication cartridge.

In an embodiment, the injection system further comprises a cartridge insertion door formed in the housing that provides access to the load tray.

In an embodiment, an auto-injector comprises a medication cartridge a housing with a loading tray configured to accept the medication cartridge, a double-sided needle configured to dispense medication from the medication cartridge, a sheath form on an injection end of the housing, wherein the sheath can be extended and retracted by a sheath spring, a telescoping plunger, and a generator, configured to impart a downward force on the telescoping plunger in order to transfer the force on the medication cartridge for dispensation of the medication from the medication cartridge into the needle.

In an embodiment, the double-sided needle comprises a first pointed end, a second pointed end, and a connective region capable of transporting medication between the first end and the second end.

In an embodiment, the medication cartridge further comprises a cap capable of being pierced by the double-sided needle.

In an embodiment, the auto-injector further comprises a removable safety cap configured to prevent the plunger from imparting force on the medication cartridge.

In an embodiment, the sheath further comprises a needle aperture.

In an embodiment, the auto-injector further comprises a first sliding stop and a second sliding stop, wherein the first sliding stop and second sliding stop restrict movement of the medication cartridge between the first sliding stop and the second sliding stop.

In an embodiment, the auto-injector further comprises a needle hub configured to hold the needle component. In an embodiment, the auto-injector further comprises a needle switch operably connected to the needle hub.

In an embodiment, a modular injector system comprises a dispenser assembly comprising: an outer housing unit, a telescoping plunger in the outer housing unit, an injection spring, configured to impart a downward force on the telescoping plunger, and a protective sheath connected to the telescoping plunger to control the initiation of the injection process; and a cartridge assembly comprising: an inner housing unit with a loading tray configured to accept a medication cartridge, and a needle configured to dispense medication from the medication cartridge to an injection site.

In an embodiment, the dispenser assembly is configured to connect to the cartridge assembly. In an embodiment, the protective sheath is retracted in order to initiate an injection.

It will be appreciated that variations of the above-disclosed and other features and functions, or alternatives thereof, may be desirably combined into many other different systems or applications. Also, it should be appreciated that various presently unforeseen or unanticipated alternatives, modifications, variations, or improvements therein may be subsequently made by those skilled in the art which are also intended to be encompassed by the following claims.

What is claimed is:

1. An injection system comprising:
a housing with a loading tray configured to accept a medication cartridge;
a needle configured to transport medication from the medication cartridge to an injection site;
a sheath formed on an injection end of the housing; and
a plunger comprising at least two telescoping sections wherein the plunger is configured to impart downward force on the medication cartridge in order to release medication in the medication cartridge.

2. The injection system of claim 1 wherein the needle further comprises:
a two sided needle comprising:
a first pointed end;
a second pointed end; and
a body connecting the first pointed end and second pointed end.

3. The injection system of claim 2 wherein one of the first pointed end and the second pointed end of the two sided needle is configured to pierce the medication cartridge.

4. The injection system of claim 1 further comprising:
a sheath spring configured to extend and retract the sheath.

5. The injection system of claim 1 further comprising:
a generator configured to impart a downward force on the plunger.

6. The injection system of claim 1 further comprising:
a removable safety cap configured to prevent the plunger from imparting downward force on the medication cartridge.

7. The injection system of claim 1 further comprising:
a needle hub configured to hold the needle.

8. The injection system of claim 7 further comprising:
a needle switch operably connected to the needle hub wherein the needle hub holds the needle in alignment with the medication cartridge.

9. The injection system of claim 1 further comprising:
a cartridge insertion door formed in the housing that provides access to the load tray.

10. An auto-injector comprising:
a medication cartridge;
a housing with a loading tray configured to accept the medication cartridge;
a double-sided needle configured to dispense medication from the medication cartridge;
a sheath form on an injection end of the housing, wherein the sheath can be extended and retracted by a sheath spring;
a telescoping plunger; and
a generator, configured to impart a downward force on the telescoping plunger in order to transfer the force on the medication cartridge for dispensation of the medication from the medication cartridge into the needle.

11. The auto-injector of claim 10 wherein the double-sided needle comprises:
a first pointed end;
a second pointed end; and
a connective region capable of transporting medication between the first end and the second end.

12. The auto-injector of claim 11 wherein the medication cartridge further comprises:
a cap capable of being pierced by the double-sided needle.

13. The auto-injector of claim 11 further comprising:
a removable safety cap configured to prevent the plunger from imparting force on the medication cartridge.

14. The auto-injector of claim 11 wherein the sheath further comprises:
a needle aperture.

15. The auto-injector of claim 11 further comprising:
a first sliding stop; and
a second sliding stop, wherein the first sliding stop and second sliding stop restrict movement of the medication cartridge between the first sliding stop and the second sliding stop.

16. The auto-injector of claim 11 further comprising:
a needle hub configured to hold the needle component.

17. The auto-injector of claim 16 further comprising:
a needle switch operably connected to the needle hub.

18. A modular injector system comprising:
a dispenser assembly comprising:
an outer housing unit;
a telescoping plunger in the outer housing unit;
an injection spring, configured to impart a downward force on the telescoping plunger; and
a protective sheath connected to the telescoping plunger to control the initiation of the injection process; and
a cartridge assembly comprising:
an inner housing unit with a loading tray configured to accept a medication cartridge; and
a needle configured to dispense medication from the medication cartridge to an injection site.

19. The modular injector system of claim 18 wherein the dispenser assembly is configured to connect to the cartridge assembly.

20. The modular injector system of claim 18 wherein the protective sheath is retracted in order to initiate an injection.
